(19) United States
(12) Patent Application Publication
(10) Pub. No.: US 2024/0215911 A1
(43) Pub. Date: Jul. 4, 2024

(54) SYSTEM AND METHOD FOR MULTIMODAL BIOLOGICAL FEEDBACK MEASUREMENT

(71) Applicant: Vilnius Gediminas Technical University, Vilnius (LT)

(72) Inventors: Arturas SERACKIS, Vilnius (LT); Kristina DAUNORAVICIENE, Vilnius (LT); Andzela SESOK, Vilniaus raj. (LT); Rytis MASKELIUNAS, Kauno raj. (LT); Vytautas ABROMAVICIUS, Vilnius (LT); Julius GRISKEVICIUS, Vilnius (LT)

(21) Appl. No.: 18/148,586

(22) Filed: Dec. 30, 2022

(51) Int. Cl. A61B 5/00; A61B 5/0205; A61B 5/11

(52) U.S. Cl. CPC A61B 5/486; A61B 5/0205; A61B 5/1116; A61B 5/7267; A61B 5/742; A61B 5/746; A61B 2560/0223

(57) ABSTRACT

This invention presents a novel multimodal system and method for monitoring and enhancing physical performance of a user. The system measures physiological and biomechanical signals such as EEG, EMG and ECG and uses machine learning algorithms to calibrate the sensors. Anomaly detection is enabled by a fuzzy logic system and alerts both the user and their doctor if necessary. The system provides visual or other feedback to the user to help improve performance and compare current and historical data to determine any changes in user.

### FIELD OF THE INVENTION

The present invention relates to a system and method for monitoring and enhancing human performance, comprising a set of sensors and tracking devices for measuring physiological and biomechanical signals, motion sensors, and human pose tracking technology for providing feedback and if an anomaly is detected, the system alerts both the user and their doctor, and provides warnings.

### BACKGROUND OF THE INVENTION

The system described as a tool for monitoring and enhancing human performance with a sensors and tracking devices that measure various physiological and biomechanical signals, motion sensors, and human pose tracking technology. The systems and methods that uses information to provide feedback to the user, monitoring of human activity using a combination of different sensors available for the user and enhancing human performance via biological feedback, are known.

U.S. Ser. No. 10/258,259 (B1)—2019 Apr. 16, Multimodal sensory feedback system and method for treatment and assessment of disequilibrium, balance and motion disorders. This invention is a system and method for measuring the biomechanical state of a subject using sensors and providing visual exercises for rehabilitation and assessment of balance and motion disorders. The biomechanical state is measured while the subject performs a predetermined task, and any variance exceeding threshold triggers feedback to the subject in the form of visual, vibrotactile, or auditory feedback. Integrates information collected from different sensors, therefore multimodal. However, it is focused on monitoring specific defects (motion distorters); next to other sensors, uses a depth sensor, designed to provide feedback related to poor posture, detection of good or bad state is based on threshold value, applied to statistical features.

US2006282003 (A1)—2006 Dec. 14, Breath biofeedback system and method. A system providing target breathing patterns and displaying images based on thoracic volume data to encourage a subject to modify respiration. It includes a thoracic volume input module, a pattern module, and a display generator. The display generator produces displayable images with a first object at a first position determined by the thoracic volume data, and a second object at a second position determined by the target breathing pattern and thoracic volume data, encouraging the subject to modify respiration. Here a specific set of signals is measured, the result is presented only through the display. Measured parameters (specific). However, there is no: universality, adaptation to other sensors, other measured parameters, etc.; no danger monitoring and user warning; doctor does not participate in the decision-making process.

US2006253052 (A1)—2006 Nov. 9, Electromyographic (EMG) feedback during AMES treatment of individuals with severe neuromuscular deficits. Method of rehabilitating a patient suffering from partial or total loss of motor control involves providing feedback in the form of a signal related to EMG activity while attempting to move the affected joint. This feedback is intended to assist the patient in developing overt movement at the joint and ultimately improve functionality. Only specific sensor identified in this patent is EMG and feedback is provided by a special device that can move the limb.

WO2022146271 (A1)—2022 Jul. 7, A participation assessment system based on multimodal evaluation of user responses for upper limb rehabilitation. This invention relates to a system that uses a multimodal sensor fusion method to evaluate a patient's participation and performance during upper extremity rehabilitation exercises and adjusts the difficulty of the exercises according to the patient's performance. The system is low-cost and can be applied to any kind of rehabilitation exercises, as well as conventional therapy and sports exercises. It also evaluates patient tiredness and slacking, which can affect therapy performance. Although it has signs of universality, it is independent of the type of devices used, in this patent, movements are evaluated by calculated angles between joints.

WO2019086997 (A2)—2019 May 9, Wearable biofeedback system. A biofeedback system with an insole and a sensory output device; insole has sensing and processing units; sensing unit monitors person's movement; processing unit performs gait analysis and determines when to provide feedback; communication unit sends indication of feedback via short range wireless communication; sensory output device outputs the feedback. The uniqueness of this patent is the use of a specific sensor in the insole. An insole sensor is used.

WO2016028220 (A1)—2016 Feb. 25, Method and apparatus for assisting movement rehabilitation. A method and apparatus for providing tele-rehabilitation with remote supervision via wireless telecommunication is disclosed. Sensors are used to motion-sense a user's body to be exercised and a second part not intended to be moved. Real-time biofeedback alerts the user of undesirable movements sensed. Data is wirelessly transmitted, processed and stored for visual representation of the exercise. Video recordings are captured for therapist review. Video conferencing allows real-time guidance. Data of multiple users can be transmitted to a therapist simultaneously. A warning is given to the user about the specific monitored situation.

The difference between described invention and the previous examples is that our system is focused on one-dimensional, time-series sensor signals, the type of feedback is not limited in advance. The decision is made by machine learning models selected from a specially prepared set, pre-trained. The size of the set can be expanded, the models are updated as the system is improved, the selection of a specific machine model for application is made taking into account what data was collected from the sensors (taking into account what sensors were used and what parameters/characteristics were calculated from them). Universality, adaptation to other sensors, other measured parameters, etc., here is danger monitoring and user warning, also the doctor participate in the decision-making process.

In our system, greater versatility is achieved by introducing a unified structure for collecting additional information. For example, this patent additionally attempts to evaluate aspects of the psychological state by measuring Heart Rate, skin conductance. In our solution, these would simply be additional digital twins that would associate these parameters with a likely psychological state, her estimate. However, our system can be supplemented with additional digital twins if it becomes possible to measure state with other sensors, such as EEG sensors, and to develop a digital twin model that relates EEG signals to an estimate of psychological state.

Our system may contain more parameters (they are not specifically evaluated), for example, it can be measured the amplitude of the movement, the range of angle values, its change, compared to historical data, register the movement of individual joints (decide whether the movement was compensated or not by not calculating the angle, but by evaluating the joint change of coordinates, because it is not always easy to calculate the angle in an uncalibrated system, when data is received from mutually uncalibrated sensors).

Also, our system includes a block/functionality for automatic sensor calibration (signal interpretation) using machine learning models, which avoids tuning the system before measurement.

### SUMMARY OF THE INVENTION

This invention is a system and method designed to monitor and enhance the physical performance of user. It includes sensors and tracking devices that measure physiological and biomechanical signals such as EEG, EMG and ECG signals. Motion sensors and human pose tracking technology are also integrated to provide detailed information about the user's body movements and posture. The system uses this data to provide feedback to the user in the form of auditory, visual, or tactile stimuli to help them improve their performance. The system also uses machine learning algorithms to calibrate the sensors, enabling it to monitor the users' vitals more accurately. If an anomaly is detected, the system alerts both the user and their doctor, and provides visual warnings to the user if necessary.

The present invention includes a universal system that combines:
automatic sensor calibration based on the application of specialized (for a specific sensor or for a specific group of sensors) machine learning algorithms, when desired/required parameter values are predicted from sensor signals (e.g. accelerometer, gyroscope and magnetometer sensor signals are sent to a machine learning model, which outputs predicted quaternion values, which are analytically difficult to calculate and the machine learning model is used as an alternative to commonly used adaptive filters).
parallel functioning anomaly detection sub-system and parameter estimation sub-system, which allow:
to inform the user about a dangerous (or potentially dangerous) change in parameters, if the alarm signal is formed by a fuzzy logic system, evaluating the rules linking the parameter values with the type of alarm signal and the percentage risk estimate and:
   a) the history of previously calculated parameters for the user,
   b) the doctor's new or historical parameters assessment data,
   c) detected anomaly (its type, in which signal it was detected, etc.).
to submit data to the biological feedback interpretation system, the purpose of which is to link the measured parameters with the conformity of a specific human subsystem (muscle condition, heart condition, etc.) to the norm, to evaluate positive or negative changes by comparing the current state with historical data.
a system for interpreting measured parameters relevant to biological feedback, which provides measured or predicted parameter values to digital twins (a specific digital twin is selected according to the type of parameters to be measured—those digital twins whose input requires the measurement of all types of required parameters are used), and we receive feedback from digital twins a connection (biological feedback) that describes the state of some human subsystem.
a block for presenting the state of the newly evaluated human subsystem to the user, which can:
compare the current state with historical data,
provide current status,
if necessary, provide a warning to the user visually (if there is a display) or in an alternative way (vibration, sound, light flashes, etc., if a specialized or suitable display is not provided in the system).

### BRIEF DESCRIPTION OF THE DRAWINGS

FIG. 1 shows an example of a preferred embodiment of the invention, depicting a person in a room with mounted sensors.

FIG. 2 shows a schematic representation of a system.

FIG. 3 shows a schematic representation of all components including a machine learning and signal processing.

FIG. 4 shows a schematic representation of a machine learning based sensor data transformation sub-system.

FIG. 5 shows a schematic representation of user's performance evaluation and its process algorithm.

FIG. 6 shows a schematic representation process of communication between a system of compatible parameters and a digital twin.

FIG. 7 shows a schematic representation of give alert and feedback.

### DETAILED DESCRIPTION OF THE INVENTION

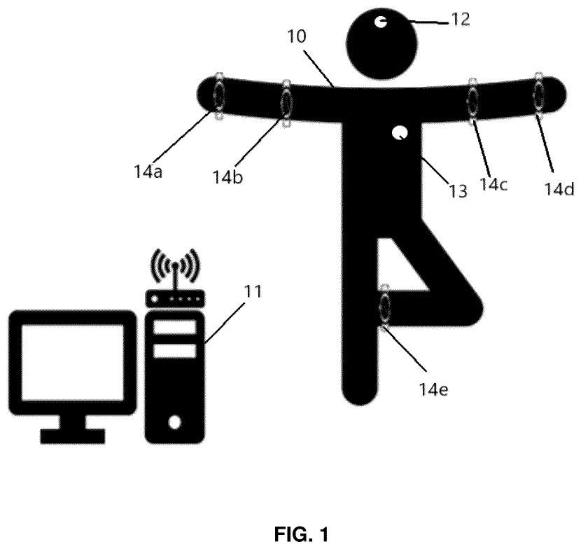

Example of use: the system shown in FIG. 1 is a tool for monitoring and enhancing user performance. A user/patient 10 with a history of heart problems is wearing devices that are monitoring his heart rate and other vitals, includes a user 10 with a set of sensors and tracking devices that can measure various physiological and biomechanical signals, such as electroencephalography (EEG) signals—electroencephalograph 12, electromyography (EMG) signals—electromyographs 14a, 14b, 14c, 14d, 14e, and electrocardiography (ECG) signals—electrocardiograph 13. The system also can include motion sensors and human pose tracking technology, which can provide detailed information about the user's body movements and posture. Described system, can detect any changes or anomalies in the patient's vitals and alert both the patient and their doctor. System uses information to provide biological feedback to the user. This can take the form of auditory, visual, or tactile stimuli that are designed to help the user improve their performance. For example, the system could provide feedback to help the user control their heart rate, regulate their breathing, or improve their posture and body alignment. The feedback can be tailored to the individual user and can be adjusted based on their specific needs and goals. The system includes a hub/computer 11 that processes the measured signals and determines appropriate feedback to provide to the user 10. This allows for automated evaluation of the user's performance, as well as personalized feedback that is tailored to the individual user 10. The system also uses machine learning algorithms to automatically calibrate the sensors, enabling it to monitor the users 10 vitals more accurately. The system can then interpret the patient's vitals and provide feedback on the state of their heart, comparing the current state with historical data. If the system detects an anomaly, it alerts to the user and their doctor, and provides visual warnings to the user if necessary.

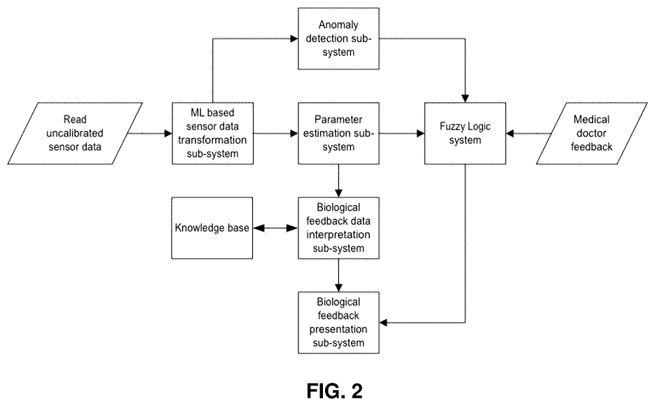

The scheme shown in FIG. 2 involves a multi-stage process that begins with reading uncalibrated sensor data. This data is then machine learning (ML) processed based sensor data transformation sub-system which can be used to identify anomalies in the data. The data is then processed by fuzzy logic system, which is used to estimate parameters, this system is combined with medical doctor feedback to arrive at a biological data interpretation sub-system. Finally, this data is presented to the medical doctor in the form of a knowledge-based feedback presentation sub-system. The fuzzy logic system is used to ensure accuracy and consistency in the data interpretation and feedback presentation.

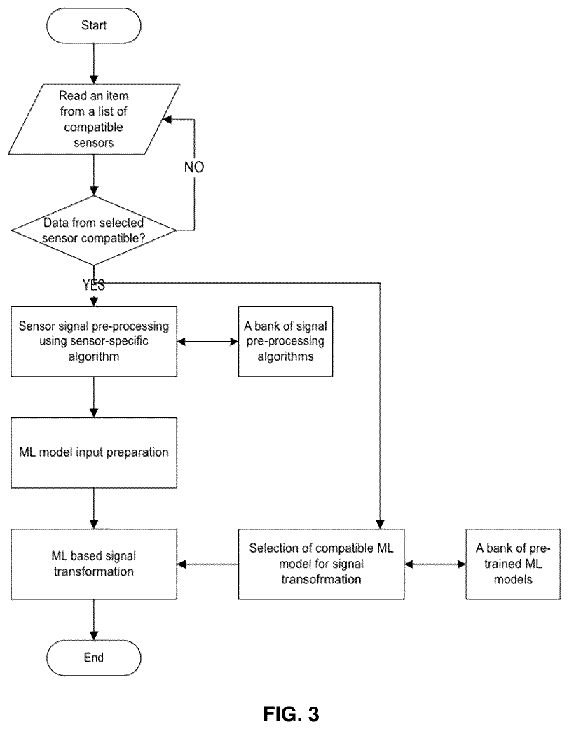

FIG. 3 scheme is designed to help identify and process data from compatible sensors. First, a list of compatible sensors is read. If data is received from a compatible sensor, the signal pre-processing is done using a sensor-specific algorithm. This is followed by a bank of signal pre-processing algorithms, which prepares the input for a machine learning (ML) model. The ML based signal transformation is then used to select a compatible ML model for signal transformation. Finally, the transformed signal is used to generate an output.

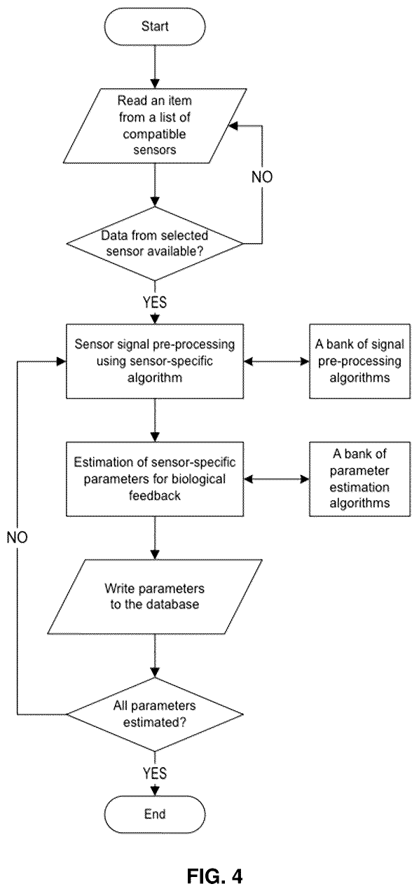

FIG. 4 shows a machine learning based sensor data transformation sub-system, used as an alternative to analytical sensor data pre-processing techniques. The idea of input-output mapping using high flexibility machine learning model (e.g., deep learning based artificial neural network structure) is applied in this sub-system. A deep learning model is trained to map raw sensor signals to calibrated values of an alternative measurement system, which gives more stable results for parameter estimation. In one of embodiments, the input signals to this sub-system could be signals from three axis of accelerometer and signals from three axis of gyroscope sensor. The output of this sub-system could be the predicted values of quaternion for current position (state) of IMU sensor. A deep learning model, selected for this signal input-output mapping task should include memory feature. A recurrent neural network units with various modifications, such as Gated Recurrent Unit, Long Short-Time Memory, could be selected as a basis of deep learning model structure.

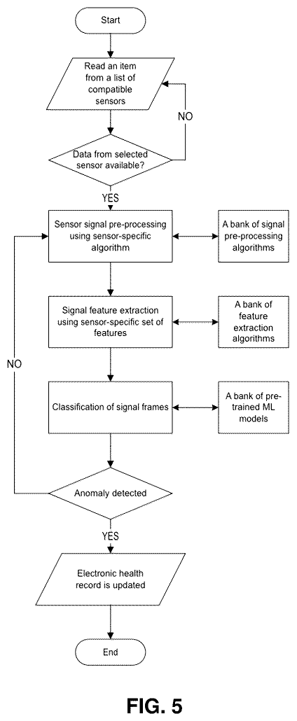

The scheme shown in FIG. 5 is designed to monitor a patient's health in real-time. It begins by reading an item from a list of compatible sensors which provides data from the selected sensor. This data is then pre-processed using a sensor-specific algorithm that is related to a bank of signal pre-processing algorithms. After pre-processing, signal feature extraction is performed using a sensor-specific set of features that are related to a bank of feature extraction algorithms. The signal frames are then classified using a bank of pretrained ML models. If an anomaly is detected, the electronic health record is automatically updated. This scheme provides a comprehensive way to monitor a patient's health in real-time and can contribute to early detection of medical issues.

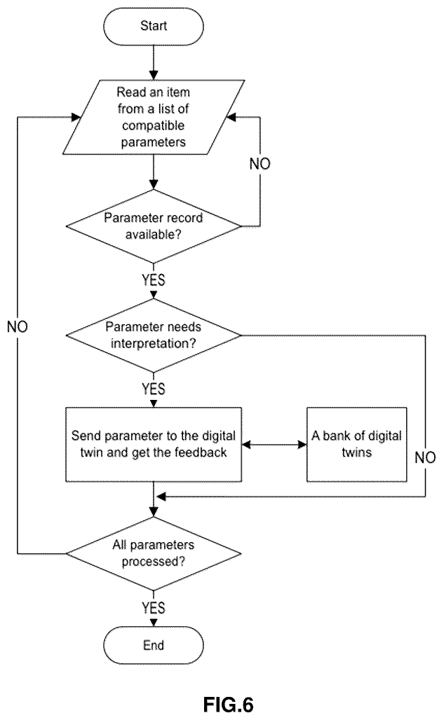

The scheme FIG. 6 shows a process of communication between a system of compatible parameters and a digital twin. The process begins with reading an item from a list of compatible parameters. If the parameter record is available, it is then interpreted and sent to the digital twin. The feedback from the digital twin is then recorded and checked to see whether all parameters are processed. If not, the process begins again by reading an item from the list of compatible parameters. Finally, the system records the feedback from the digital twin and stores it in a bank of digital twins.

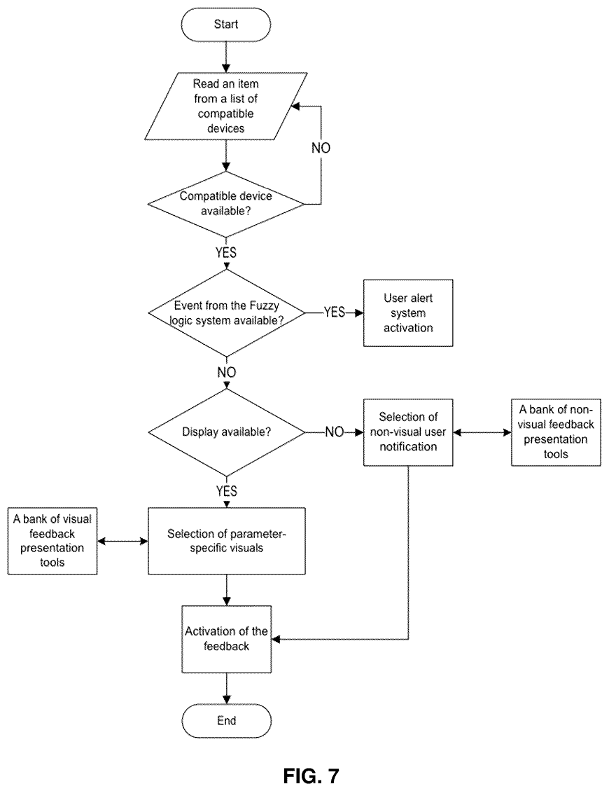

This scheme FIG. 7 is designed to alert and give feedback for users. If a display is available visually and otherwise gives non-visual feedback.

The system can be used for a wide range of applications, including sports training, rehabilitation, and more. For example, the system could be used to help athletes improve their performance by providing feedback on their physiological and biomechanical signals. The system could also be used in rehabilitation settings to help patients recover from injuries or conditions that affect their movement and function.

1. System for multimodal biological feedback measurement, comprising: a user with a set of sensors and tracking devices that can measure various physiological and biomechanical signals; motion sensors; human pose tracking technology; a hub/computer that processes the measured signals; and machine learning algorithms to automatically calibrate the sensors.

2. A system of claim 1, further providing measured or predicted parameter values to digital twins and receiving feedback from the digital twins in the form of a connection describing the state of user subsystem.

3. The system of claim 1, further comprising feedback components to provide biological feedback to the user, the feedback components comprising one or more of auditory, visual, or tactile stimuli.

4. The system of claim 3, wherein the feedback is tailored to the individual user and can be adjusted based on their specific needs and goals.

5. The system of claim 1, wherein the system further includes the ability to interpret the patient's vitals and provide feedback on its state, comparing the current state with historical data.

6. The system of claim 1, wherein if the system detects an anomaly, it alerts to the user and their doctor, and if necessary, provides visual warnings to the user.

7. Method for multimodal biological feedback measurement, comprising:
providing a user with a set of sensors and tracking devices configured to measure various physiological and biomechanical signals, such as electroencephalography (EEG) signals, electromyography (EMG) signals, and electrocardiography (ECG) signals;
providing motion sensors and human pose tracking technology;
using machine learning algorithms to automatically calibrate the sensors, enabling it to monitor the user's vitals more accurately;
detecting any changes or anomalies in the user's vitals and alerting both the user and their doctor;
providing biological feedback to the user in the form of auditory, visual, or tactile stimuli designed to help the user improve their performance;
processing using digital twins to interpret the measured parameters relevant to the biological feedback and determining appropriate feedback to provide to the user;
interpreting the user's vitals and providing feedback, comparing the current state with historical data.
